(19) United States
(12) Patent Application Publication
(10) Pub. No.: US 2007/0172821 A1
(43) Pub. Date: Jul. 26, 2007

(54) Assembly of chitosan onto an electrode surface

(76) Inventors: Li-Qun Wu, N. Potomac, MD (US); Mark J. Kastantin, Goleta, CA (US); Hyunmin Yi, Beltsville, MD (US); Gary W. Rubloff, Clarksville, MD (US); William E. Bentley, Clarksville, MD (US); Reza Ghodssi, Rockville, MD (US); Gregory F. Payne, Hunt Valley, MD (US)

Correspondence Address: EDELL, SHAPIRO & FINNAN, LLC, 1901 RESEARCH BLVD, SUITE 400, ROCKVILLE, MD, 20850-3164, US

(21) Appl. No.: 10/525,241

(22) PCT Filed: Aug. 22, 2003
(86) PCT No.: PCT/US03/26356

§ 371 Date: Jan. 8, 2007

Related U.S. Application Data
(60) Provisional application No. 60/405,582, filed on Aug. 23, 2002.

(51) Int. Cl. C12Q 1/68; H01L 21/00; C12M 3/00

(52) U.S. Cl. 435/6; 204/471; 435/287.2; 427/2.11; 438/1; 257/E51.02

(57) ABSTRACT

The deposition of chitosan onto electrode surfaces is disclosed. Methods of depositing chitosan on surfaces are disclosed. Materials comprising chitosan deposited on a substrate are also disclosed.

This application claims the benefit of U.S. provisional application No. 60/405,582, filed Aug. 23, 2002, the entirety of which is incorporated herein by reference. The U.S. government may have certain rights to this invention, pursuant to Grant No. BES-0114790, awarded by the National Science Foundation.

### 1. FIELD OF THE INVENTION

The invention relates to methods of depositing polysaccharide chitosan from a chitosan solution onto a substrate.

### 2. BACKGROUND OF THE INVENTION

The ability to create devices (e.g., biosensors, microarrays, and micro electromechanical systems (MEMS)) requires facile methods to precisely control surfaces. A variety of patterning techniques can be used to produce desired structures, while various methods have been investigated to control surface chemistries. For instance, surface chemistries have been controlled by self-assembling monolayers using reactions between thiols and metal surfaces, or between alkyltrichlorosilanes and oxidized silicon. Bain, C. D., Whitesides, G. M. Angew. Chem. Int. Ed. Engl. 1989, 28, 506-512; Whitesides, G. M., Laibinis, P. E. Langm. 1990, 6, 87-96; Sagiv, J. J. Am. Chem. Soc. 102, 1980, 92-98; Brzoska, J. B., Azouz, I. B.; Rondelez, F. Langm. 1994, 10, 43 67-4373; Allara, D. L., Parikh, A. N., Rondelez, F. Langm. 1995, 11, 2357-2360. An additional method to assemble macromolecules and particles is to exploit an applied voltage. Foo, G. M., Pandey, R. B. Biomacromol. 2000, 1, 407-412. Applied voltages have been used to assemble colloidal particles, proteins, and cells onto electrode surfaces. Bohmer, M. Langm. 1996, 12, 5747-5750; Strike, D. J., Rooij, N. F., de Koudelka-Hep, M. Biosen. Bioelect. 1995, 10, 61-66; Cosnier, S. Biosen. Bioelect. 1999, 14, 443-456; Kurzawa, C., Hengstenberg, A., Schuhmann, W. Anal. Chem. 2002, 74, 355-361; Kurzawa, C., Hengstenberg, A., Schuhianan, W. Anal. Chem. 2002, 74, 355-361; Brisson, V., Tilton, R. D. Biotechnol. Bioeng. 2002, 77, 290-295.

Chitosan is an amine-rich polysaccharide derived by deacetylation of chitin. Chitin is the second most abundant polysaccharide in nature and is found in crustaceans, insects, and fungi. Chitosan is becoming an increasingly important biopolymer because it offers unique physicochemical properties. Hudson, S. M.; Smith, C. In Biopolymers from Renewable Resources, D. L. Kaplan (Ed.), Springer, Berlin, 1998, p. 96-118. Specifically, chitosan has primary amino groups that have pKa values of about 6.3. Rinaudo, M., Pavlov, G., Desbrieres, J. Polymer 1999, 40, 7029-7032; Sorlier, P., Denuziere, A., Viton, C., Domard, A. Biomacromolec. 2001, 2, 765-772. At pHs below the pKa, most of the amino groups are protonated making chitosan a water-soluble, cationic polyelectrolyte. Chitosan's water-solubility is unique as other β,(1→4)-linked polysaccharides (e.g., cellulose and chitin) are insoluble. At pHs above the pKa, chitosan's amino groups are deprotonated, and this polymer becomes insoluble. Chitosan's pH-dependent solubility is attractive because it allows processing from aqueous solutions while a modest increase in pH to neutrality enables chitosan to be formed into various shapes (e.g., beads, membranes, and films). Ligler, F. S., Lingerfelt, B. M., Price, R. P., Schoen, P. E. Langm. 2001, 17, 5082-5084. An additional feature is that chitosan's amino groups confer nucleophilic properties to this polymer. Specifically, the deprotonated amino groups have an unshared electron pair that can undergo reaction with a variety of electrophiles. As a result, various chemistries can be exploited to crosslink chitosan and to graft substituents onto this polymer. Hirano, S., Ohe, Y., Ono, H. Carbohydr. Res. 1976, 47, 315-320; Muzzarelli, R. A. A., Taniani, F., Emanuelli, M., Marioth, S. Carbohydr Res. 1982, 107, 199-214; Yalpani, M., Hall, L. D. Macromol. 1984, 17, 272-281; Roberts, G. A. F., Taylor, K. E. Die Makromolek. Chemie. 1989, 190, 951 - 960; Hsien, T.-Y., Rorrer, G. L. Sep. Sci. Technol. 1995, 30, 2455-2475; Gruber, J. V., Rutar, V., Bandekar, J., Konish, P. N. Macromolec. 1995, 28, 8865-8867; Xu, J., McCarthy, S. P., Gross, R. A., Kaplan D. L. Macromolec. 1996. 29, 3436-3440; Knaul, J. Z., Hudson, S. M., Creber, K. A. M. J. Polym. Sci.: B: Polym. Phys. 1999, 37, 1079-1094; Mi, F.-L., Kuan, C. Y., Shyu, S.-S., Lee, S. T., Chang, S. F. Carbohydr. Polym. 2000, 41, 389-396; Mi, F.-L., Sung, H.-W., Shyu, S.-S. J. Appl. Polym. Sci. 2001, 81, 1700-1711; Kurita, K., Ikeda, H., Yoshida, Y., Shimojoh, M., Harata, M. Biomacromolec. 2002, 3, 1-4.

### 3. SUMMARY OF THE INVENTION

The invention encompasses methods of depositing a thin layer of the polysaccharide chitosan onto the surface of an electrode substrate. The methods comprise contacting the substrate with a chitosan solution and applying an electric current to the substrate. The invention also encompasses substrates onto which a layer of chitosan has been deposited.

### 3.1 FIGURES

Various aspects of the invention may be understood with reference to the following figures:

FIG. 1 represents a diagram of chitosan deposition;

FIG. 2 provides a graphical representation of the deposition of chitosan onto the surface of an electrode, wherein deposition occurred from a 1 w/v % chitosan solution using an applied voltage of 2.5 V;

FIG. 3 provides an SEM micrograph of a deposited layer on an electrode (a) without neutralization and (b) with neutralization;

FIG. 4 represents deposition under the following conditions, each of which include immersing the electrode in caustic, rinsing it extensively and drying it prior to measuring the thickness: (a) deposition occurring from a 1 w/v % chitosan solution using an applied voltage of 2.5 V; (b) deposition measured after 6 minutes using chitosan solutions of varying concentrations and an applied voltage of 2.5 V; (c) deposition measured after 6 minutes from a 1 w/v % chitosan solution using varying voltages;

FIG. 5 provides an IR spectrum of deposited material and chitosan, wherein the material deposited on the electrode was neutralized in base, extensively washed with distilled water, and dried; the IR spectrum was collected using a KBr pellet; and the control spectrum was collected using a chitosan film; and

FIG. 6 provides an ES-MS spectrum of deposited material after incubation with chitosanase.

### 4. DETAILED DESCRIPTION OF THE INVENTION

As used herein and unless otherwise indicated, a “substrate” is a material upon which chitosan can be deposited. Suitable substrates are electrically conducting, and are made of materials such as, but not limited to, metals (e.g., aluminum, antimony, cadmium, chromium, cobalt, copper, gold, iron, lead, magnesium, mercury, nickel, palladium, platinum, silver, steel, tin, tungsten, zinc, and alloys thereof) semiconductors, and conductive polymers.

As used herein and unless otherwise indicated, a “cell” may be eucaryotic or prokaryotic and may be from any source where cells can be obtained.

For the chitosan solution used to deposit chitosan onto a substrate, suitable concentrations of chitosan vary from about 0.0001 to about 0.001 (w/v) %, about 0.001 to about 0.01 (w/v) %, about 0.01 to about 0.1 (w/v) %, about 0.1 to about 1 (w/v) %, about 1 to about 10 (w/v) %, about 10 to about 20 (w/v), and about 20 to about 30 (w/v) %. A suitable pH for deposition of chitosan onto a substrate is any pH where chitosan remains soluble and in solution. It is further recognized that the concentration of the chitosan solution, the voltage and the time a current is applied to deposit chitosan onto a substrate can be varied to control the extent of chitosan deposition.

In a specific embodiment of the present invention, the method of depositing chitosan onto a metal substrate comprises: a) contacting the substrate with a solution containing chitosan; and b) applying an electric current to the substrate, sufficient to deposit chitosan onto the substrate. In another specific embodiment, the method of depositing chitosan onto a metal substrate further comprises washing the substrate containing deposited chitosan with at least one liquid selected from the group consisting of water, a solution with neutral pH, a basic solution and an acidic solution. In another specific embodiment, the method of depositing chitosan onto a metal substrate further comprises contacting the chitosan-bound substrate with chitosanase.

A specific embodiment of the present invention is a substrate coated with chitosan. In a particular embodiment, the thickness of the chitosan is from about 0.01 to about 3 microns, from about 0.01 to about 1.5 microns, or from about 0.02 to about 0.8 microns.

A further specific embodiment is a substrate coated with chitosan further comprising bound protein. Another specific embodiment is a substrate coated with chitosan further comprising a bound enzyme. Another specific embodiment is a substrate coated with chitosan further comprising bound polynucleotide. Yet another specific embodiment is a substrate coated with chitosan further comprising either bound RNA or DNA. Still another specific embodiment is a substrate coated with chitosan further comprising bound cells. A further specific embodiment of the inventions is a substrate coated with chitosan wherein the substrate is a metal.

### 5. EXAMPLE

Chitosan from crab shells (85% deacetylation as reported by the supplier) and the enzyme chitosanase were purchased from Sigma-Aldrich Chemicals. Chitosanase was reported by the manufacturer to have specific activities of 102.3 U/mg. Chitosan solutions were prepared by adding chitosan flakes to water and incrementally adding small amounts of HCl to the solution to maintain the pH near 3. After filtering undissolved material, these chitosan solutions were diluted to various concentrations, and the pH was adjusted to 5.0 using NaOH (1 M).

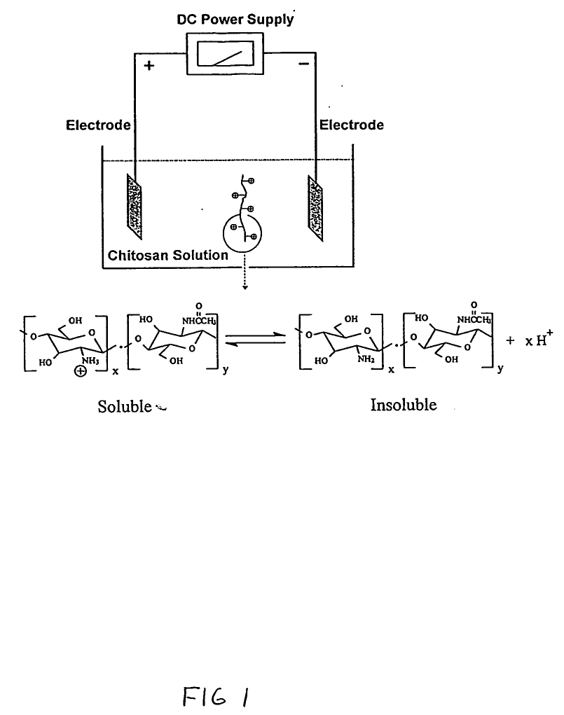

Electrodes were prepared by depositing 90 Å thick chromium (Cr) and then 2000 Å thick gold (Au) films on 4-inch diameter silicon wafers already coated with 1 -micron thick thermal oxide film. For chitosan deposition, the electrodes were dipped into a chitosan solution (pH=5, 1% w/v) as shown in FIG. 1. In most experiments, three electrodes were examined. Two of the electrodes (positive and negative) were connected to a DC voltage supply using alligator clips. The third electrode was not connected to a power supply and is designated a “neutral” electrode. At specific times the electrodes were removed from the solution and rinsed with distilled water, after which the voltage was removed. In some cases, electrodes were immediately oven-dried (60° C. for 3 hours). In other cases, electrodes were neutralized by immersion in a basic solution (1 M NaOH) and then rinsed with distilled water prior to drying. After drying, the thickness of the deposited layers was measured by a profilometer (ALPHA-STEP 500 SURFACE PROFILER, TENCOR Instruments).

Thicknesses were measured on different areas of the electrode surface and the average values were calculated.

Scanning electron microscopy (SEM) was used to study the surface morphology of the deposited layer. SEM micrographs have been recorded using a Focused Ion Beam system (FIB/SEM workstation dual beam 620; FEI Company). Samples on silicon substrates were placed in the chamber having vacuum of about 10⁻⁶ Torr. Structural properties were examined at a 20,000-fold magnification.

For chemical analysis, deposition was obtained by placing electrodes in a chitosan bath (1 w/v %; pH=5) for 20 minutes with an applied voltage of 2.0 volts. For IR analysis, the negative electrode was removed from the chitosan solution, rinsed, disconnected from the power supply, and then placed in about 1 M NaOH overnight. When the electrode was soaked in base for such a long time, the deposited material was observed to detach from the electrode surface. This deposited material was then extensively washed with distilled water and dried overnight at 60° C. After drying, it was ground with KBr powder and pressed into a pellet. IR spectra were collected using a Perlin-Elmer 2000 FT-IR system.

For analysis by electrospray mass spectrometry (ES-MS), the negative electrode was removed from the chitosan solution, rinsed, disconnected from the power supply, and then placed in a small volume of dilute acid (HCl; pH=3) and held overnight to allow the deposited material to dissolve. This acid solution was recovered, diluted to approximately 0.08 w/v % and the pH was adjusted to 5.5. The sample was then incubated for one day at 37° C. with the enzyme chitosanase (0.2 U/ml). After incubation the solution was filtered to remove precipitates, and analyzed by ES-MS (Thermo Finnigan, San Jose, Calif., USA). All samples for ES-MS analysis were diluted in an aqueous solution containing 0.1% formic acid and analyzed in positive ion mode.

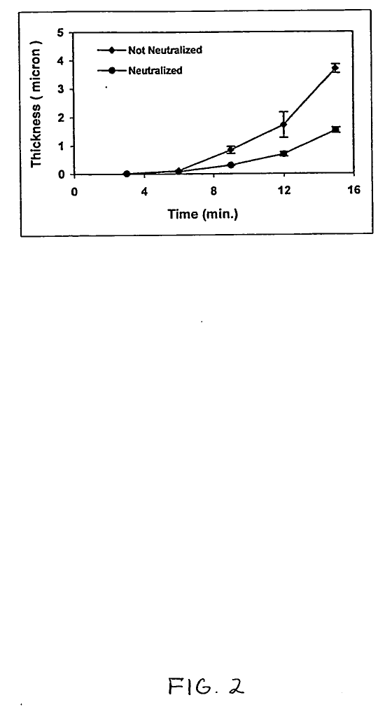

To examine deposition, we immersed electrodes in an acidic chitosan solution and applied a voltage of 2.5 V. After applying the voltage for varying times, negative electrodes were removed from the solution, rinsed with distilled water, and the voltage was removed. In some cases, the electrodes were dried, while in other cases they were immersed in base, rinsed and then dried. After drying, the thickness of the deposited layer was measured by profilometry. FIG. 2 shows that the thickness of the deposited layer increases over time. Additionally, FIG. 2 shows that the thickness of the deposited layer is less when the electrode was treated with base.

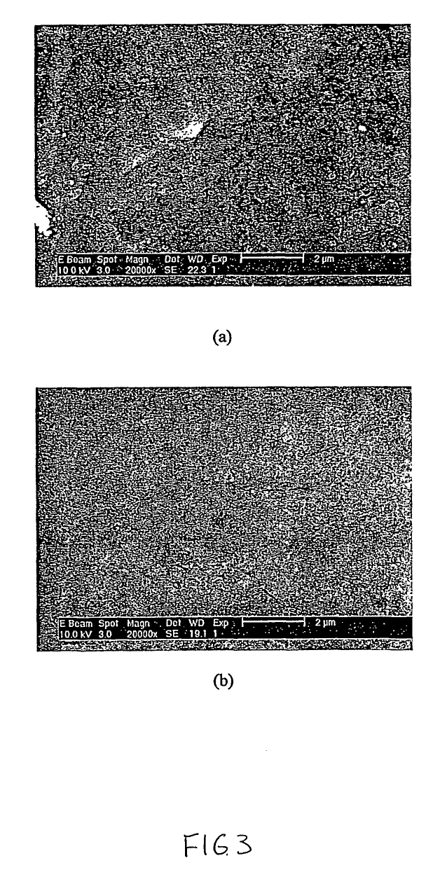

Scanning electron microscopy (SEM) was used to examine the surface morphology of the negative electrodes. FIG. 3a shows micrographs for electrodes that were dried without neutralization. As can be seen from FIG. 3a, this sample has a non-uniform surface morphology. Possibly, the surface roughness of this electrode may be due to the presence of salts associated with the chitosan polyelectrolyte. FIG. 3b shows the surface of a negative electrode that had been immersed in base and rinsed extensively before drying. As indicated in FIG. 3b, the surface of this electrode is more uniform—presumably due to the neutralization of chitosan. The observation in FIG. 2 that deposited layers are thinner after neutralization suggests that neutralization leads to a collapse of the polymer network and possibly also the elimination of salts. In subsequent experiments, neutralization was performed prior to measuring the thickness of deposited layers.

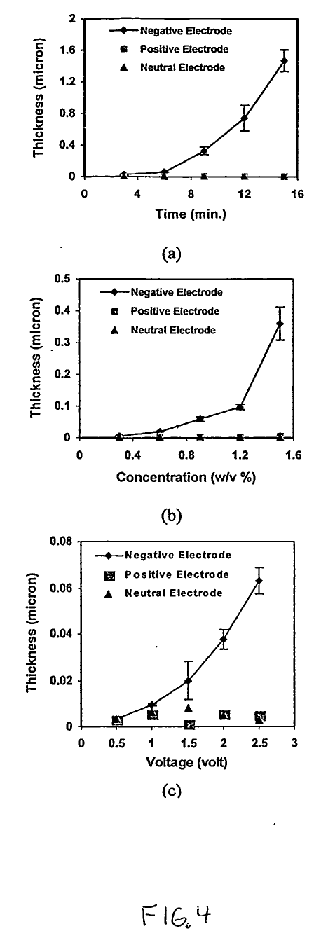

Additional studies were performed to characterize deposition, and to compare deposition onto the negative and positive electrodes. FIG. 4a shows that the thickness of the deposited layer on the negative electrode increased over time. No material was observed to deposit on the positive electrode under the conditions studied. An additional control was an electrode in which no voltage was applied (designated as “neutral” electrode). As shown in FIG. 4a, no deposition was observed on the surface of this “neutral” electrode. FIG. 4b shows that when the concentration of chitosan in the solution was increased, there was increased deposition on the surface of the negative electrode. Again no deposition was observed on the positive electrode or on the control electrode in which no voltage was applied. FIG. 4c shows that deposition on the negative electrode also increased with increasing voltage.

In summary, FIGS. 2 through 4 demonstrate that an applied voltage can be used to deposit a thin layer onto a negative electrode when the electrode is immersed in a chitosan solution. Additionally, the thickness of the deposited layer can be controlled by the deposition conditions. Finally, once the deposited layer is neutralized, it can be retained on the electrode surface even in the absence of an applied voltage (i.e., the electrode can be extensively rinsed). This latter observation is consistent with the fact that chitosan is insoluble under neutral and basic conditions.

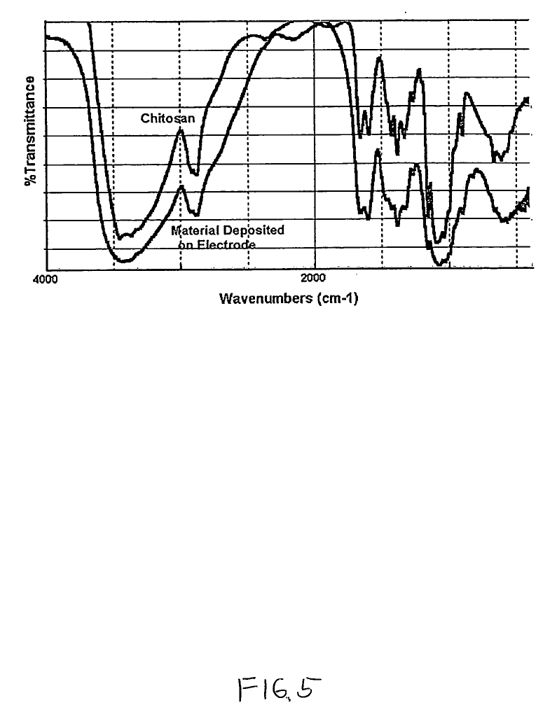

Two independent techniques were used to provide chemical evidence that the material deposited on the negative electrode is chitosan. For IR analysis, the “neutralized” material was recovered from the electrode surface, rinsed extensively, dried overnight, and incorporated into a KBr pellet. FIG. 5 compares the IR spectrum for the KBr pellet of the deposited material with the spectrum of a chitosan film. The absorption spectra are similar for the two samples providing evidence that the material deposited on the negative electrode is chitosan. Some differences in the spectra are observed in the amine and amide regions (1500-1700 cm⁻¹ ) suggesting the possibility that chitosan chains that are more highly deacetylated (and therefore more highly charged) may be preferentially deposited onto the negative electrode. Sannan, T., Kurita, K., Ogura, K., Iwakura, Y. Polymer 1978, 19, 458-459; Domszey, J. G., Roberts, G. A. F. Makromol Chem. 1985, 186, 1671-1677; Shigemasa, Y., Matsuura, H., Sashiwa, H., Saimoto, H. Int. J. Biol. Macromol. 1996, 18, 237-242.

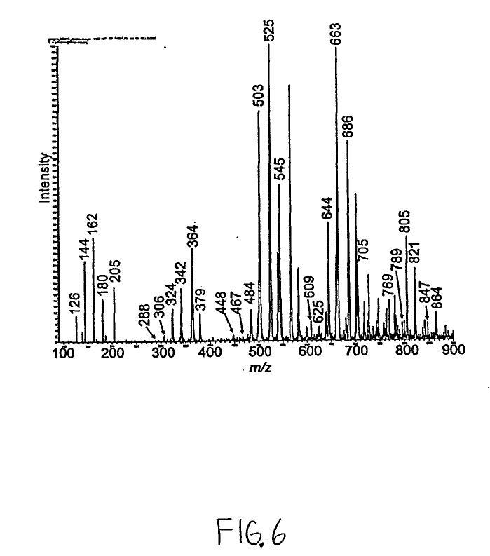

The second technique to provide chemical evidence that the deposited material is chitosan was provided by electrospray mass spectrometry (ES-MS). Because chitosan's molecular weight (>300,000 g/mol) exceeds the limit for analysis, we enzymatically hydrolyzed the deposited material and analyzed the hydrolysate. For this analysis, the deposited layer was dissolved from the electrode surface into an acidic solution. After dilution, the solution was incubated with the chitosan-hydrolyzing enzyme, chitosanase. Osswald, W. F., McDonald, R. E., Nied, R. P., Shapiro, J. P., Mayer, R. T. Anal. Biochem. 1992, 204, 40-46. FIG. 6 shows the ES-MS results for this hydrolyzate.

To examine the results in FIG. 6, we considered the peaks expected for the enzymatic hydrolysis of chitosan. Enzymatic hydrolysis of chitosan is known to result in the formation of various species (e.g., monomers, dimers). Shahgholi, M., Callahan, J. H., Rappoli, B. J., Rowley, D. A. J. Mass Spectrom. 1997, 32, 1080-1093. Additionally, chitosan is a copolymer of glucosamine and N-acetylglucosamine, and the predominant oligomeric species are expected to consist of either glucosamine units or both glucosamine and N-acetylglucosamine units. Because the degree of acetylation is low (15%), it is not expected that significant amounts of oligomers that contain more than a single N-acetylglucosamine residue. Finally, it is known that MS spectra of glucosamine and glucosamine trimers contain product ions resulting from the loss of H₂O. Kerwin, J. L., Whitney, D. L., Sheikh, A. Insect Biochem. Molec. 1999, 29, 599-607. Table 1 lists a series of peaks expected for the hydrolysis of chitosan (e.g., various monomers, dimers, trimers, tetramers, and pentamers). By comparison of these expectations with results in FIG. 6 (listed in parenthesis in Table 1), it is clear that the ES-MS provides strong evidence that the deposited material is chitosan.

A control in the ES-MS study was provided by a sample that was incubated in the absence of chitosanase. The ES-MS analysis of this control showed weak signals with a low signal-to-noise ratio (not shown). This is consistent with the expectation that un-hydrolyzed chitosan will be too large (300,000 g/mol) to be measured by ES-MS. The highest signals in this control appeared at m/z of 220 and 299 and the latter signal does not even appear in FIG. 6. Thus, without being limited by theory, chitosanase-catalyzed hydrolysis of the deposited material may be necessary to attain strong signals in the ES-MS.

TABLE 1
Expected and observed m/z values for enzymatically hydrolyzed
chitosan. (Observed values from FIG. 6 appear in parenthesis)

| | Mono- | | Tri- | Tetra- | Penta- |
|---|---|---|---|---|---|
| | mer | Dimer | mer | mer | mer |
| (Gln)ₓ-3H₂O | 126 | 287 | 448 | 609 | 770 |
| | (126) | (288) | (448) | (609) | (769) |
| (Gln)ₓ-2H₂O | 144 | 305 | 466 | 627 | 788 |
| | (144) | (306) | (467) | (625) | (789) |
| (Gln)ₓ-H₂O | 162 | 323 | 484 | 645 | 806 |
| | (162) | (324) | (484) | (644) | (805) |
| (Gln)ₓ | 180 | 341 | 502 | 663 | 824 |
| | (180) | (342) | (503) | (663) | (821) |
| [GlcNAc•(Gln)ₓ₋₁]-H₂O | 204 | 365 | 526 | 687 | 848 |
| | (205) | (364) | (525) | (686) | (847) |
| [GlcNAc•(Gln)ₓ₋₁] | 222 | 383 | 544 | 705 | 866 |
| | | | (545) | (705) | (864) |

Gln: Glucosamine;
GlcNAc: N-Acetylglucosamine.

In summary, two independent techniques were used to provide chemical evidence that the deposited material was chitosan. Standard IR analysis shows that the spectrum for the deposited material is similar to the spectrum for chitosan. Further, the deposited material was susceptible to hydrolysis by the enzyme chitosanase while the hydrolysate shows a family of peaks consistent with glucosamine and N-acetylglucosamine residues—the repeating units of chitosan.

Chitosan is a unique biopolymer that offers interesting possibilities for controlling the surface chemistry of devices. First, chitosan is an amine-rich polysaccharide that is positively charged under mildly acidic conditions. This characteristic allows a thin chitosan layer to be deposited (i.e., “self-assembled”) onto a negative electrode in response to an applied voltage. The results reported here demonstrate that the thickness of the deposited layer can be controlled by the conditions used. Second, chitosan's pKa is rather low (pKa≈6.3) compared to other amine-containing biopolymers (e.g., polylysine's pKa is 10.5), and above it's pKa chitosan is insoluble. As a result of this pH-dependent solubility, a simple neutralization step is sufficient to convert chitosan to an insoluble form that can be retained on the surface of the electrode (i.e., the applied voltage is only required for deposition and not to retain the chitosan layer). Third, the high content of primary amine groups allows a chitosan coating to be used for controlling surface properties and for subsequent modification steps. The utility of amine groups is illustrated by the current interest in creating amine-terminated monolayers. Whitesides, G. M., Laibinis, P. E. Langm. 1990, 6, 87-96; Gole, A., Sainkar, S. R., Sastry, M. Chem. Mater. 2000, 12, 1234-1239; Sieval, A. B., Linke, R., Heij, G., Meijer, G., Zuilhof, H., Sudholter, E. J. R. Langm. 2001, 17, 7554-7559; Wallwork, M. L., Smith, D. A., Zhang, J., Kirkham, J., Robinson, C. Langm. 2001, 17, 1126-1131; Nishiyama, K., Kubo, A., Ueda, A., Taniguchi, I. Chem. Lett. 2002, (1), 80-81; Jiang, X., Ortiz, C., Hammond, P. T. Langm. 2002, 18, 1131-1143. The amine groups also enable biologically active molecules (e.g., peptides and proteins) to be coupled onto chitosan surfaces using standard coupling chemistries (e.g., glutaraldehyde- or carbodiimide- based chemistries) or using enzymatic methods. Vazquez-Duhalt, R., Tinoco, R., D'Antonio, P., Topoleski, L. D. T., Payne G. F. Bioconj. Chem., 2001, 12, 301-306. Finally, chitosan is gaining increasing attention as a biomaterial for applications ranging from enzyme immobilization to the creation of biocompatible surfaces. Airoldi, C., Monteiro, O. A. C. J. Appl. Polym. Sci. 2000, 77, 797-804; Belmonte, M. M., De Benediftis, A., Muzzarelli, R. A. A., Mengucci, P., Biagini, G., Gandolfi, M. G., Zucchini, C., Krajewski, A., Ravaglioli, A., Roncari, E., Fini, M., Giardino, R. J.Mater. Sci.-Mater. Med. 1998, 9, 485-492; Lvov, Y., Onda, M., Ariga, K., Kunitake, T. J, Biomat. Sci.—Polym. Ed., 1998, 9, 345-355; Wang, D. A., Ji, J., Sun, Y. H., Yu, G. H., Feng, L. X. J. Biomed. Mater. Res. 2001, 58, 372-383; Gong, H. P., Zhong Y. H., Li, J. C., Gong, Y. D., Zhao, N. M., Zhang, X. F. J. Biomed. Mater. Res. 2000, 52, 285-295. Thus, chitosan may provide an appropriate interface between biological systems and microelectronic devices.

The prior example is provided as illustration of the disclosed invention and is not intended to limit the scope of the invention. All cited references are herein incorporated in their entireties by reference.

We claim:

1. A method of depositing chitosan onto a substrate, comprising:
a) contacting the substrate with a solution containing chitosan; and
b) applying an electric current to the substrate sufficient to deposit the chitosan onto the substrate.

2. The method of claim 1, further comprising washing the substrate containing deposited chitosan with water, a solution with a neutral pH, a basic solution, or an acidic solution.

3. The method of claim 1, further comprising contacting chitosan deposited on the substrate with chitosanase.

4. The method of claim 1, wherein the substrate is a semiconductor.

5. The method of claim 1, wherein the substrate is a conductive polymer.

6. The method of claim 1, wherein the substrate is a metal.

7. The method of claim 1, wherein the solution contains chitosan in a concentration of from about 0.0001 to about 30% w/v.

8. The method of claim 7, wherein the solution contains chitosan in a concentration of from about 0.1 to about 10% w/v.

9. A material obtained by the method of claim 1.

10. A material comprising chitosan deposited on a substrate.

11. The material of claim 10, wherein the substrate is a metal, a semi-conductor, or a conductive polymer.

12. The material of claim 11, wherein the substrate is a metal.

13. The material of claim 12, wherein the metal is aluminum, antimony, cadmium, chromium, cobalt, copper, gold, iron, lead, magnesium, mercury, nickel, palladium, platinum, silver, steel, tin, tungsten, zinc, or an alloy thereof.

14. The material of claim 10, further comprising a protein bound to the cbitosan.

15. The material of claim 10, further comprising an enzyme bound to the chitosan.

16. The material of claim 10, further comprising a polynucleotide bound to the chitosan.

17. The material of claim 16, wherein the bound polynucleotide is RNA.

18. The material of claim 16, wherein the bound polynucleotide is DNA.

19. The substrate of claim 10, further comprising cells bound to the chitosan.
